(19) United States
(12) Patent Application Publication
(10) Pub. No.: US 2010/0000413 A1
(43) Pub. Date: Jan. 7, 2010

(54) AIR FILTRATION DEVICE

(75) Inventors: Mark Francis Turner, Mechanicsville, VA (US); Martin Patrick Brady, Richmond, VA (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: HAMILTON BEACH BRANDS, INC., GLEN ALLEN, VA (US)

(21) Appl. No.: 12/165,897

(22) Filed: Jul. 1, 2008

(51) Int. Cl. A61L 9/04; A61L 9/12; B01D 46/10

(52) U.S. Cl. 96/222; 55/471; 55/410

(57) ABSTRACT

An air filtration device configured for operational association with an electrical outlet mounted on a wall includes a housing the defines a front surface, a rear surface and an air flow path therethrough. A cover is attached to the front surface of the housing in a spaced-apart relationship. The cover and the front surface of the housing define a slot. An impeller is provided for moving air through the air flow path. A motor drives the impeller. The motor and the impeller are supported within the housing. A tray is removably positionable within the slot.

### BACKGROUND OF THE INVENTION

The present invention relates broadly to air filtration apparatus and, more particularly, to an air filtration device that is configured for abutment against an electrical outlet providing operational power for the device, with atmospheric air flowing into an intake on the front surface of the device, and filtered air being emitted through vents provided on the top of the device.

As the atmosphere in general becomes more polluted and the general population becomes more aware of discomforts associated with dirty air, air filtration devices of all shapes, types and sizes have become more popular. One type of air filtration device provides a small fan for use in a smoky environment. These devices typically will include an electric fan contained in a small housing for tabletop use, with a filter through which the air is directed for removal of smoke and other such contaminants. These small fans are useful for removing odors and contaminants from a confined space, since they typically take up little space themselves and move a relatively small volume of air.

Such current designs, while effective, leave room for improvement in the areas of noise control, air dispersion and filter management.

### SUMMARY OF THE INVENTION

Briefly stated, a preferred embodiment of the present invention is directed to an air filtration device for intake of atmospheric air, removal of contaminants from the atmospheric air, and expulsion of filtered air. The device is configured for operational association with a generally vertically oriented surface. The air filtration device includes a housing defining an air flow path therethrough. The housing includes a front surface and a rear surface. A cover is attached to the front surface of the housing. An impeller is provided for moving air through the air flow path. A motor drives the impeller. The motor and the impeller are supported within the housing. A tray is removably positionable between the housing and the cover in the air flow path. The tray includes a filter and a scent cartridge.

In another aspect, a preferred embodiment of the present application is directed to an air filtration device configured for operational association with an electrical outlet mounted on a wall. The air filtration device includes a housing defining a front surface, a rear surface and an air flow path therethrough. A cover is attached to the front surface of the housing in a spaced-apart relationship. The cover and front surface of the housing define a slot. An impeller is provided for moving air through the air flow path. A motor drives the impeller. The motor and the impeller are supported within the housing. A tray is removably positionable within the slot.

### BRIEF DESCRIPTION OF THE DRAWINGS

The foregoing summary, as well as the following detailed description of a preferred embodiment of the present invention, will be better understood when read in conjunction with the appended drawings. For the purpose of illustrating the invention described in the present application, there is shown in the drawings, an embodiment which is presently preferred. It should be understood, however, that the invention is not limited to the precise arrangements and instrumentalities shown. In the drawings:

FIG. 1 is a top front perspective view of an air filtration device according to a preferred embodiment of the present invention;

FIG. 2 is a top rear perspective view of the air filtration device illustrated in FIG. 1, with a tray in a partially removed position;

FIG. 3 is a rear right perspective view of the air filtration device illustrated in FIG. 1, with the tray in an almost fully removed position; and

FIG. 4 is an exploded perspective view of the air filtration device illustrated in FIG. 1.

### DETAILED DESCRIPTION OF THE INVENTION

Certain terminology is used in the following description for convenience only and is not limiting. The words “right,” “left,” “lower,” “upper,” “front” and “rear” designate directions in the drawings to which reference is made. The words “inwardly” and “outwardly” refer to directions toward and away from, respectively, the geometric center of the air filtration device and designated parts thereof. The terminology includes the above-listed words, derivatives thereof and words of similar import.

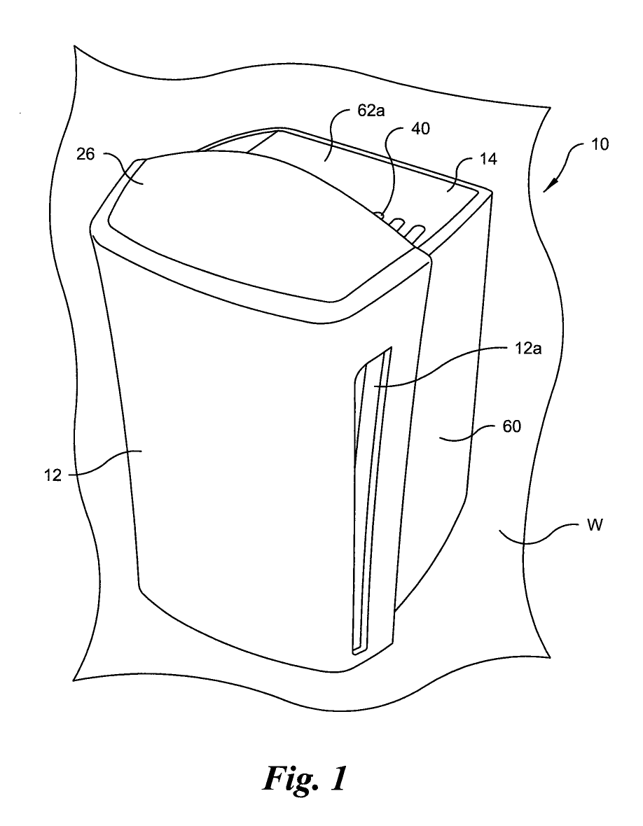

Referring now to the drawings in detail, wherein like elements indicate like elements throughout, there is shown in FIGS. 1-4 a presently preferred embodiment of an air filtration device, an air diffuser or an air purifier, generally designated 10, for intake of atmospheric air, removal of contaminants from the atmospheric air, and expulsion or emission of filtered air. The air filtration device 10 is configured for operational association with a generally vertically surface, such as an electrical outlet E mounted on a wall W.

The air filtration device 10 includes a housing 14 defining an air flow path therethrough. The housing 14 includes a front surface 14a and rear surface 14b. The front surface 14a is spaced-apart from the rear surface 14b by a pair of sidewalls 60 and top and bottom walls 62a, 62b. A generally hollow space or cavity 14c is defined between the front surface 14a, the rear surface 14b, the sidewalls 60 and the top and bottom walls 62a, 62b of the housing 14. The top wall 62a of the housing 14 includes a plurality of spaced-apart vents 40 between the front surface 14a and the rear surface 14b. The vents 40 preferably function as an air outlet for the air filtration device 10. However, those skilled in the art understand that the vents could alternatively function as an air inlet if rotation of an impeller 24 within the housing 14 is reversed. The vents 40 extend generally parallel to the sidewalls 60 and are open at the front surface 14a of the housing 12. The air outlet is not limited to the inclusion of the above-described vents 40, but may alternatively include virtually perforated layer without departing from the broad inventive concept of the present invention.

The rear surface 14b of the housing 14 includes an opening 18 to permit at least one but preferably a pair of electrical prongs 42 to extend from a motor 22 of the air filtration device 10 into the electrical outlet E. It is understood by those skilled in the art that the air filtration device 10 may include three electrical prongs 42, such as polarized or non-U.S. electrical connections. The rear surface 14b of the housing 14 cooperates with the generally vertical surface, or electrical outlet E mounted on the wall W, to which the air filtration device 10 is associated for enhanced dispersion of filtered air expelled through the air outlet or plurality of vents 40. The housing 14 is preferably formed of a lightweight, high strength material, such as a polymeric material. However, it is understood by those skilled in the art that virtually any material, such as a metallic material, can be used to construct the housing 14.

In the preferred embodiment, a generally arcuate-shaped cover 12 is attached to the front surface 14a of the housing 14 in a generally spaced-apart manner or relationship. The cover 12 defines at least one air inlet and preferably at least two spaced-apart air inlets 12a, 12b that extend in parallel to one another along a height of the cover 12 and on opposite lateral sides of the cover 12. As previously mentioned, the top wall 62a of the housing 14 defines at least one air outlet, preferably in the form of the plurality of vents 40. However, it is understood by those skilled in the art that the air inlet(s) 12a, 12b and air outlet(s) 40 of the air filtration device 10 can be reversed upon reversal of the impeller 24. Further, it is understood by those skilled in the art that the size, location and number of air inlets 12a, 12b and air outlets 40 is not limited to that shown and described herein. In the preferred embodiment, the cover 12 may either be permanently attached to the housing 14 or removably attached to the housing 14. Specifically, an inner surface of the cover 12 may include at least one tab 44, an elongated tooth (not shown) or a slot (not shown) to engage a corresponding groove, slit or slot (not shown) proximate the front surface 14a of the housing 14. Alternatively, the cover 12 may be adhesively attached to the housing 12 or attached by rivets, screws, bolts, etc.

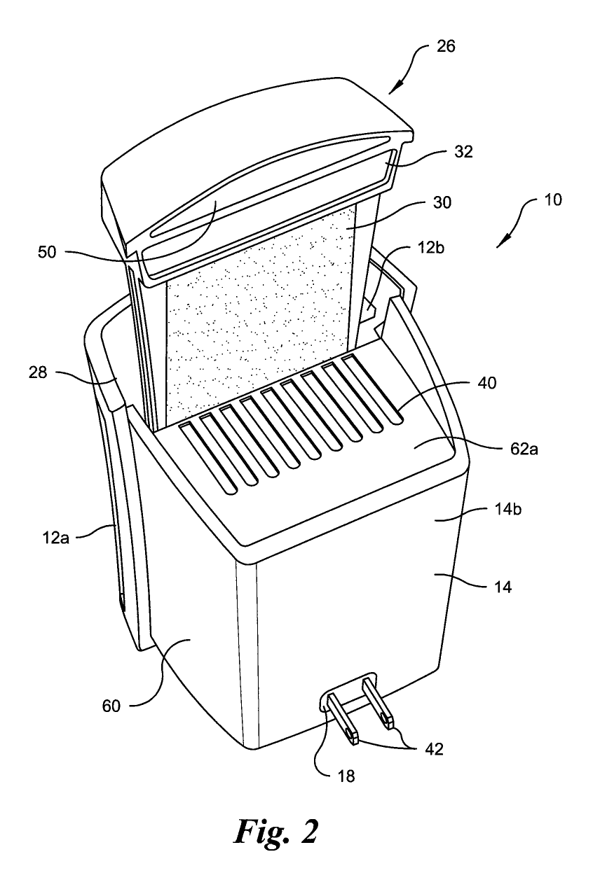

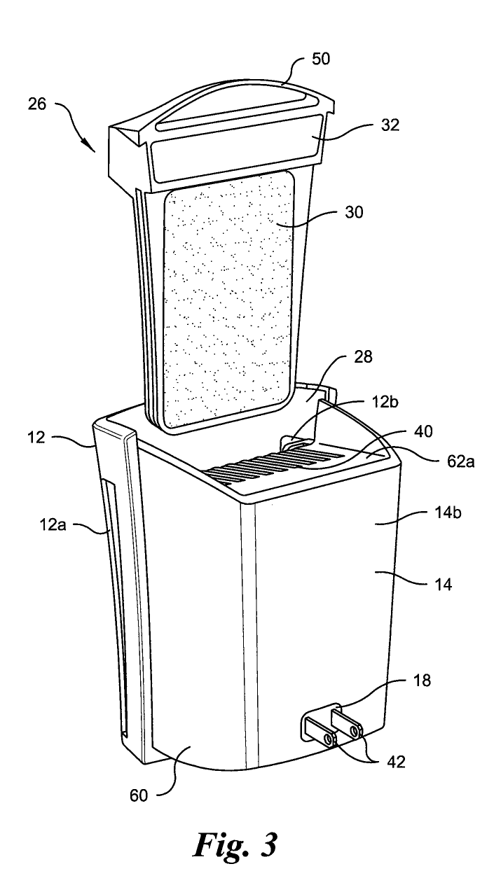

Referring to FIGS. 2 and 3, when the cover 12 is attached to the housing 14, the cover 12 and the housing 14 define a slot 28. The slot 28 is sized and shaped to receive at least a portion of a tray 26 of the air filtration device 10. The slot 28 is generally enclosed at a bottom end thereof by the combined cover 12 and housing 14 and is generally opened or exposed at an upper end of the slot 28 such that at least a filter 30 of the tray 26 can be moved into and out of the air flow path defined by the cover 12 and the housing 14.

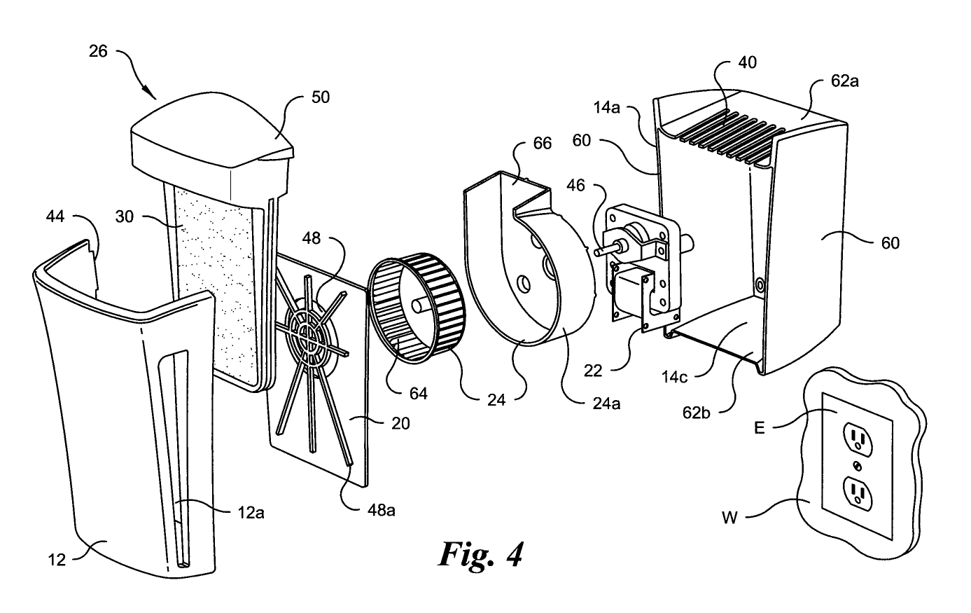

As seen in FIG. 4, an impeller 24 or fan is supported by the housing 14 for moving air through the air flow path. The impeller 24 includes a base or scroll 24a and a blade assembly 24b to be rotated within the base 24a. The blade assembly 24b is generally circular in shape and includes at least one but preferably a plurality of spaced-apart fan blades 64. The base 24a is also generally circular in shape and includes an conduit 66 designed to direct the expelled air upwardly toward the outlet vents 40 of the housing 14. The blade assembly 24b of the impeller 24 is driven by the motor 22. Both the motor 22 and the impeller 24 are supported within the cavity 14c of the housing 14. The motor 22 includes an armature or output shaft 46 that extends through the base 24a and is attached to the center of the blade assembly 24b of the impeller 24. Electrical excitation of the motor 22 causes the armature 46 to rotate, thereby rotating the blade assembly 24a of the impeller 24. The electrical prongs 42 provide electrical power to the motor 22 when the air filtration device 10 is connected to the electrical outlet E.

Specifically referring to FIG. 4, the air filtration device 10 further includes a baffle plate 20 located within the air flow path and supported by the front surface 14a of the housing 14. The baffle plate 20 is positioned between the cover 12 and the housing 14 and encloses the motor 22 and the impeller 24 within the housing 14. The baffle plate 20 includes a plurality of spaced-apart apertures 48 to allow for the passage of air therethrough. Further, a plurality of reinforcement ribs 48a increase the rigidity of the baffle plate 20.

As seen in FIGS. 1-4, the tray 26 is removably positionable between the housing 14 and the cover 12 in the air flow path. Specifically, the tray 26 is removably positionable within the slot 28 defined between the cover 12 and the housing 14. The tray 26 includes the filter 30, preferably in the form of a carbon filter, and a scent cartridge 32. The filter 30 is preferably rectangular in shape and is secured on all four sides by the tray 26. One useful filter 30 includes a front filtration surface (not shown), a rear scrim (not shown) and is charcoal activated. It will be appreciated by those skilled in the filtration art that other filter compositions may be used. In particular, some filters may be configured to remove bathroom odors and some filters may be configured to remove kitchen odors. Other types of filters may include an increased charcoal content for smoke filtration. It is contemplated that various types of filters may be used with the present air filtration device 10 without reducing the effectiveness of any of the filters or the overall filtering efficacy of the device 10. Preferably, the filter 30 is sized and shaped to cover the entire air flow path when the tray 26 is placed entirely within the slot 28. The scent cartridge 32 may be in the form of a liquid or a in a semi-solid or solid state.

When the tray 26 is fully positioned within the slot 28 (FIG. 1), the scent cartridge 32 is located above the filter 30 and the air flow path. In the preferred embodiment, the filter 30 and the scent cartridge 32 are permanently attached to the tray 26. However, it is understood by those skilled in the art that either or one of or both of the filter 30 and scent cartridge 32 are selectively removable from the tray 26 by the user to either be replaced or discarded. Further, the tray 26 may include an handle 50 integrally formed within the tray 26 above the scent cartridge 32. The handle 50 allows a user to easily and conveniently insert and remove the tray 26 from within the slot 28.

In operation, the air filtration device 10 of the present invention is plugged into the electrical outlet E using the electrical prongs 42 in a generally conventional manner. In this position, the rear surface 14b of the housing 12 is closely adjacent to the electrical outlet E and may abut the electrical outlet E and/or wall W. Once the electrical prongs 42 are connected to the electrical outlet E, power is supplied to the motor 22, which, in turn, causes the blade assembly 24b of the impeller 24 to rotate, thereby drawing atmospheric air inwardly through the air inlet(s) 12a, 12b and into the air flow path. The atmospheric air is then drawn through the filter 30 and is caused to travel along the air flow path. As the air passes through the filter 30, contaminants are removed and the filtered air travels through the apertures 48 in the baffle 20. Next, the filtered air is direct upwardly through the conduit 66 in the base 24a of the impeller and directed out of the air filtration device 10 through the vents 40. As the filtered air is directed past or through the vents 40, at least a portion of the filtered air comes in contact with some of the vapors and/or scents released from the scent cartridge 32. Specifically, the expulsion of filtered air traveling in the air flow path passes over or near the scent cartridge 32 for deodorizing the filtered air and/or dispensing a scent from the scent cartridge 32. Therefore, the air that travels through the air filtration device 10 of the present invention is both filtered and deodorized.

It will therefore be readily understood by those persons skilled in the art that the present invention is susceptible of a broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements, the present invention being limited only by the claims appended hereto and the equivalents thereof.

It will be appreciated by those skilled in the art that changes could be made to the embodiment described above without departing from the broad inventive concept thereof. It is understood, therefore, that this invention is not limited to the particular embodiment disclosed, but is intended to cover modifications within the spirit and scope of the present invention as defined by the appended claims.

I/we claim:

1. An air filtration device for intake of atmospheric air, removal of contaminants from the atmospheric air, and expulsion of filtered air, the device being configured for operational association with a generally vertically oriented surface, said air filtration device comprising:
a housing defining an air flow path therethrough, said housing including a front surface and a rear surface;
a cover attached to said front surface of said housing;
an impeller for moving air through said air flow path;
a motor for driving said impeller, said motor and impeller supported within said housing;
a tray removably positionable between said housing and said cover in said air flow path, said tray including a filter and a scent cartridge.

2. The air filtration device of claim 1, wherein said cover defines at least one air inlet and said housing defines at least one air outlet.

3. The air filtration device of claim 2, wherein said cover defines at least two spaced-apart air inlets that extend in parallel to one another.

4. The air filtration device of claim 3, wherein said air outlet is formed in a top surface of said housing.

5. The air filtration device of claim 1, wherein said cover is permanently attached to said housing.

6. The air filtration device of claim 1, wherein said cover is removably attached to said housing.

7. The air filtration device of claim 2, wherein said air outlet is formed intermediate said front and rear surfaces of said housing, said rear surface of said housing cooperating with a generally vertical surface to which said air filtration device is associated for enhanced dispersion of filtered air expelled through said air outlet.

8. The air filtration device of claim 1, said device further comprising:
a baffle plate supported by said front surface of said housing in said airflow path; said baffle plate enclosing said motor and impeller within said housing.

9. The air filtration device of claim 8, wherein said baffle plate includes a plurality of apertures to allow for the passage of air therethrough.

10. The air filtration device of claim 1, wherein said tray further includes a handle proximate said scent cartridge.

11. The air filtration device of claim 10, wherein said scent cartridge is located above said airflow path when said tray is positioned between said housing and said cover.

12. The air filtration device of claim 11, wherein said expulsion of filtered air passes over or near said scent cartridge for deodorizing said filtered air and/or dispensing a scent from said scent cartridge.

13. The air filtration device of claim 1, wherein said cover and said housing define a slot sized and shaped to receive at least a portion of said tray.

14. The air filtration device of claim 1, wherein said filter and said scent cartridge are permanently attached to said tray.

15. An air filtration device configured for operational association with an electrical outlet mounted on a wall, said air filtration device comprising:
a housing defining a front surface, a rear surface, and an air flow path therethrough;
a cover attached to said front surface of said housing in a spaced-apart relationship, said cover and said front surface of said housing defining a slot;
an impeller for moving air through said air flow path;
a motor for driving said impeller, said motor and impeller supported within said housing;
a tray removably positionable within said slot.

16. The air filtration device of claim 15, wherein said tray includes a carbon filter and a scent cartridge.

17. The air filtration device of claim 16, wherein air traveling through said air flow path passes over or near said scent cartridge for deodorizing the air and/or dispensing a scent from said scent cartridge.

18. The air filtration device of claim 15, wherein said cover defines at least two spaced-apart air inlets and said housing defines at least one air outlet.

19. The air filtration device of claim 18, wherein said air outlet is formed intermediate said front and rear surfaces of said housing, said rear surface of said housing cooperating with said wall to which said air filtration device is associated for enhanced dispersion of filtered air expelled through said air outlet.
